(19) United States
(12) Patent Application Publication
(10) Pub. No.: US 2006/0089714 A1
(43) Pub. Date: Apr. 27, 2006

(54) Intervertebral implant and rachis stabilization device

(75) Inventors: Mingyan Liu, Bourg la Reine (FR); Loic Josse, Paris (FR); Jean-Charles LeHuec, Pessac (FR); Philippe Maxy, Claye Souilly (FR)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 10/990,787

(22) Filed: Nov. 17, 2004

(30) Foreign Application Priority Data
Oct. 22, 2004 (FR) FR 04 11314

(51) Int. Cl. A61F 2/44

(52) U.S. Cl. 623/17.11

(57) ABSTRACT

An intervertebral implant has two-plates intended to come into contact with a patient's vertebral end-plates. One of the plates has a cut-out delineating webs converging toward a zone where the plates are connected to one another.

### CROSS REFERENCE

The present patent claims the benefit of a corresponding French application FR 04 11314 filed Oct. 22, 2004, entitled Intervertebral Implant and Rachis Stabilization Device, the contents of which is hereby incorporated by reference.

### FIELD OF THE INVENTION

The present invention relates to devices intended to be implanted between vertebrae, and, in some embodiments, more specifically to implants intended to replace damaged intervertebral disks.

### BACKGROUND

Implants intended for the partial or total replacement of intervertebral disks are principally used in the cervical, thoracic, lumbar and lower dorsal regions of the rachis. Some of them comprise two plates, each coming into contact with a vertebral plate, and articulated one on another by a ball joint. This type of connection has the disadvantage of giving rise to relatively intense rubbing during relative movements of the vertebrae. There is therefore a high probability of wear of the implants, resulting in an emission of debris into the patient's body, which is clearly undesirable. To avoid this, materials have to be used which have the disadvantage of incompatibility with the performance of examination by scanning or by MRI. Furthermore, this connection by ball joint alone does not bring about the possibility of the implant returning to a nominal position after a movement, a possibility which would be analogous to the elastic behavior of a natural disk.

### SUMMARY

Several embodiments of an intervertebral implant and rachis stabilization device are disclosed. In one embodiment, an intervertebral implant includes two connected plates configured to contact with opposing vertebral plates. At least one of the plates has a cut-out delineating webs converging toward a zone where the plates are connected to one another. In some embodiments, the webs are not in mutual contact when the implant is ready for use.

In another embodiment, an intervertebral implant includes two connected plates configured to contact with opposing vertebral plates. Each plate has a cut-out delineating webs converging toward a zone where the plates are connected to one another, and a hub comprising a seat and an excrescence able to penetrate and be kept in the seat for fixing said plates to one another.

In yet another embodiment, an intervertebral implant includes two connected plates configured to contact with opposing vertebral plates. A plurality of webs are included, converging toward a zone where the plates are connected to one another. A cover is also included for each plate.

Additional embodiments are disclosed in the following discussion and the included drawings. Furthermore, the claims are directed broadly to the general invention therein defined, and should not be limited to any of the disclosed embodiments.

### BRIEF DESCRIPTION OF THE DRAWINGS

The invention will be better understood on reading the following description, given with reference to the following accompanying drawings.

FIG. 1 shows, in views from below (FIG. 1a), from the front in section along B-B (FIG. 1b), from the left (FIG. 1c) and from the right (FIG. 1d), a first example of an implant according to one embodiment of the invention, composed of two assembled plates, of which the lower plate is shown in perspective seen from above in FIG. 1e and seen from below in FIG. 1f.

FIG. 2 shows in the same manner as FIG. 1 a second example of an implant according to one embodiment of the invention which is an alternative to the preceding one.

FIG. 3 shows views from below (FIG. 3a), front view in section along B-B, from the left in section along C-C, from the right, of a third example of a prosthesis according to one embodiment of the invention composed of two assembled plates, the lower plate being shown in perspective seen from above in FIG. 3e and from below in FIG. 3f.

FIG. 4 shows, in the same manner as FIG. 1, a fourth example of an implant according to one embodiment of the invention.

FIG. 5 shows, in the same manner as FIG. 1, a fifth example of an implant according to one embodiment of the invention.

FIG. 6 shows, in the same manner as FIG. 1, a sixth example of an implant according to one embodiment of the invention.

FIG. 7 shows, in the same manner as FIG. 1, a seventh example of an implant according to one embodiment of the invention.

FIG. 8 shows, in the same manner as FIG. 1, an eighth example of an implant according to one embodiment of the invention.

FIG. 9 shows, in the same manner as FIG. 1, a ninth example of an implant according to one embodiment of the invention.

FIG. 10 shows views from below (FIG. 10a), front view in section along B-B (FIG. 10b), from the left in section along C-C (FIG. 10c), from the right (FIG. 10d), and in perspective (FIG. 10e), a ninth example of an implant according to one embodiment of the invention, the upper plate (FIG. 10b) and lower plate (FIG. 10g) being shown separately in perspective.

FIG. 11 shows (offset by a quarter of turn as compared to the other similar Figs.) views from below (FIG. 11a), in section along B-B (FIG. 11b), from the left (FIG. 11c), from the right (FIG. 1d) and in perspective from above (FIG. 11e) and from below (FIG. 11f) a tenth example of an implant according to one embodiment of the invention.

FIG. 12 shows a view from below (FIG. 12a), from the front (FIG. 12b), left (FIG. 12c), and right in section along D-D, of an eleventh example of an implant according to one embodiment of the invention.

FIG. 13 shows a twelfth example of an implant according to one embodiment of the invention, in the same manner as FIG. 12.

FIG. 14 shows a thirteenth example of an implant according to one embodiment of the invention, in the same manner as FIG. 12.

FIG. 15 shows a fourteenth example of an implant according to one embodiment of the invention, in views from below (FIG. 15a), from the front and in partial section along A-A (FIG. 15b), from the left in section along C-C (FIG. 15c), and from the right (FIG. 15d).

FIG. 16 shows in perspective the prosthesis of FIG. 15 and the covers which preferably complete it.

FIG. 17 shows a fifteenth example of an implant according to one embodiment of the invention, in the same manner as in FIG. 15.

FIG. 18 shows views from below (FIG. 18a), from the front in section along B-B (FIG. 18b), and from the left (FIG. 18c), of the implant of FIG. 17 equipped with its covers.

FIG. 19 shows in perspective the separated elements of a sixteenth example of an implant according to one embodiment of the invention.

FIG. 20 shows in perspective the same example in the assembled state.

FIG. 21 shows views from below (FIG. 21a), from the left in section along B-B (FIG. 21b), from above in section along CC (FIG. 21c), from the left (FIG. 21d), from the right in section along E-E (FIG. 21e), from the front in section along F-F (FIG. 21f) and from the front in section along G-G (FIG. 21g) this same example of an implant, deprived of its covers.

FIG. 22 shows views from above (FIG. 22a), from the left in section along B-B (FIG. 22b), from below (FIG. 22c) from the rear (FIG. 22d) the lower cover of this same example of an implant.

### DETAILED DESCRIPTION

The present disclosure relates generally to the field of orthopedic surgery, and in some embodiments, to intervertebral implants. For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the invention relates. To facilitate the understanding of the invention, many of the drawings are out of scale, or include components that are out of scale. In addition, certain steps may not be shown in detail in the drawings, but are described below and are generally well known in the art. Furthermore, although reference numbers are repeated throughout the embodiments, this does not by itself dictate a relationship between the elements, but is instead provided for the sake of example.

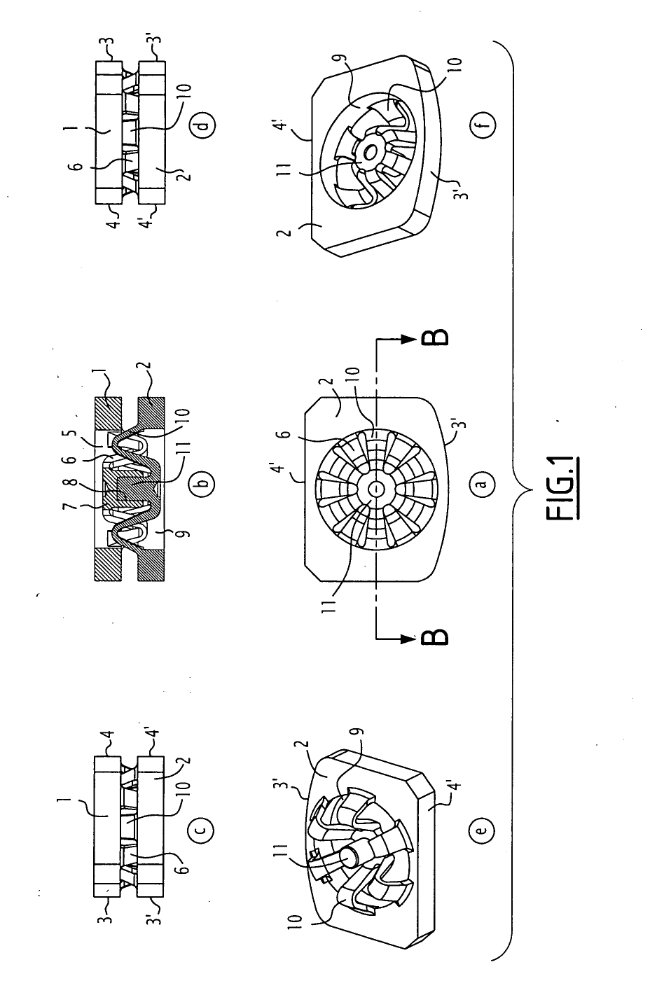

A first example of an intervertebral implant according to one embodiment of the invention is shown in FIG. 1. This implant has an upper plate 1 and a lower plate 2. The contours of these two plates 1, 2 are substantially identical in shape and dimension and are preferably, as shown, designed to substantially closely follow with their front 3, 3′ and rear 4, 4′ edges the contours of the vertebral plates.

The upper plate 1 has a circular perforation 5, within which elastic webs 6 with a substantially V-shaped cross section extend from its periphery to the center. These webs radially connect the periphery of the perforation 5 to a central hub 7 of the upper plate 1. This hub 7 is generally cylindrical in shape and has a seat 8 turned toward the lower face of the upper plate 1.

The lower plate 2 has a circular perforation 9, within which elastic webs 10 with a substantially inverted V-shaped cross section extend from its periphery to the center. These webs radially connect the periphery of the perforation 9 to a central hub 11 of the lower plate 2. This hub 11 is generally cylindrical in shape and is designed to be able to penetrate into the seat 8 of the hub 7 of the upper plate 1 with a tight fit ensuring fixing of the two pates 1, 2 to one another during the assembly of the implant before installation in a patient's intervertebral space. This tight fit may be completed or replaced by a laser weld or any other fixing mode. As may be seen in FIGS. 1a-1d, the webs 8 of the upper plate 1 and the webs 10 of the lower plate 2 are angularly mutually offset so as not to come into mutual contact when the implant is assembled.

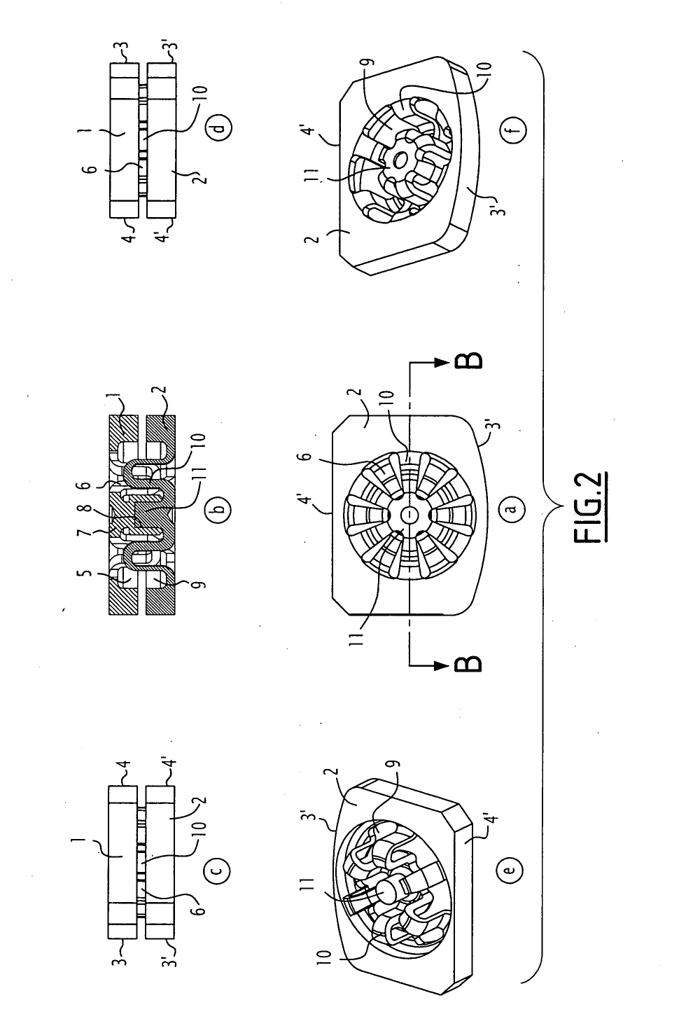

The implant shown in FIG. 2 is similar in principle to that of FIG. 1 except that the webs 6 of the upper plate have a U-shaped cross section and the webs 10 of the lower plate have a cross section of substantially inverted U-shape. It is also to be noted that the thickness of each web 6, 10 varies according to the zone of the web, so as to optimize their strength and flexibility. This feature could be found on the webs 6, 10 of all the embodiments of the invention.

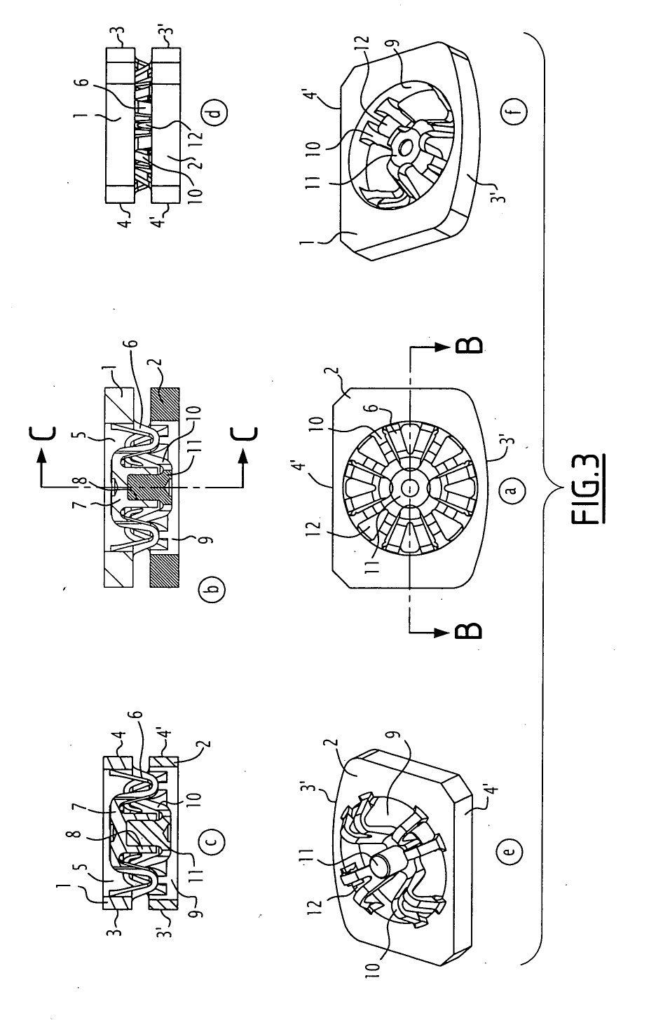

The implant shown in FIG. 3 is similar to that of FIG. 1 except that the webs 6, 10 of the two plates 1, 2 each have a cavity 12 whereby each web is attached to the corresponding hub 7, 11 in a single zone and is attached to the periphery of the corresponding perforation 8, 9 in two zones. The flexibility of the webs may thereby be increased without greatly reducing their strength.

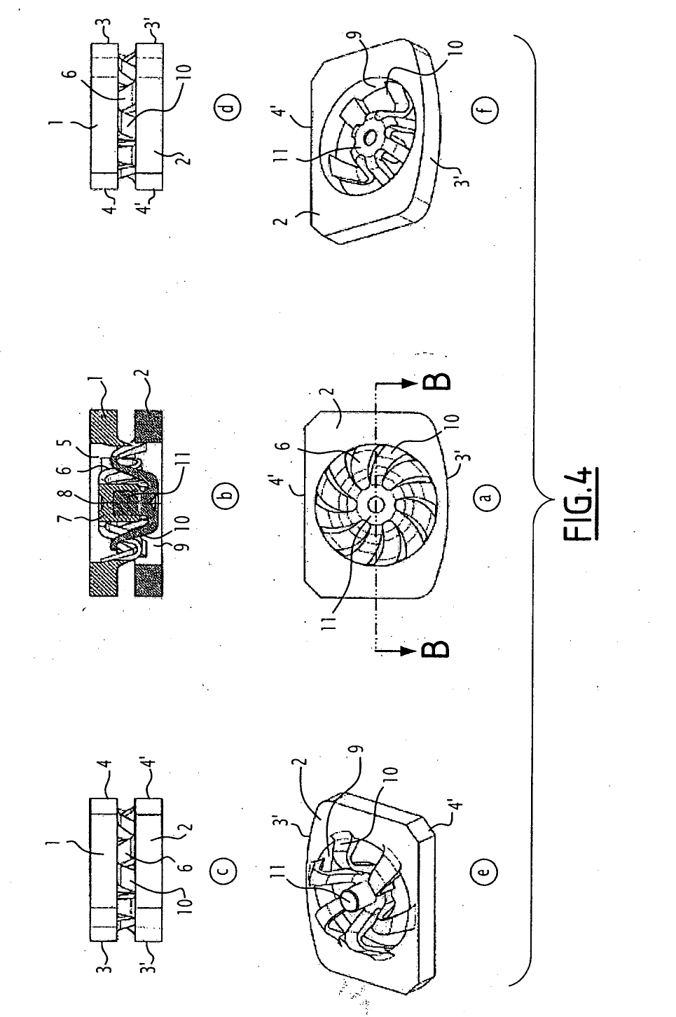

The implant shown in FIG. 4 is similar to that of FIG. 1 except that the webs 6, 10 of the two plates 1, 2 do not connect the peripheries of the perforations 5, 9 to the hubs 7, 11 radially but along an orientation having a helicoidal curvature. So, the useful fiber length for flexion can be increased. It should be understood that this latter characteristic could also be adapted to the examples of FIGS. 2 and 3.

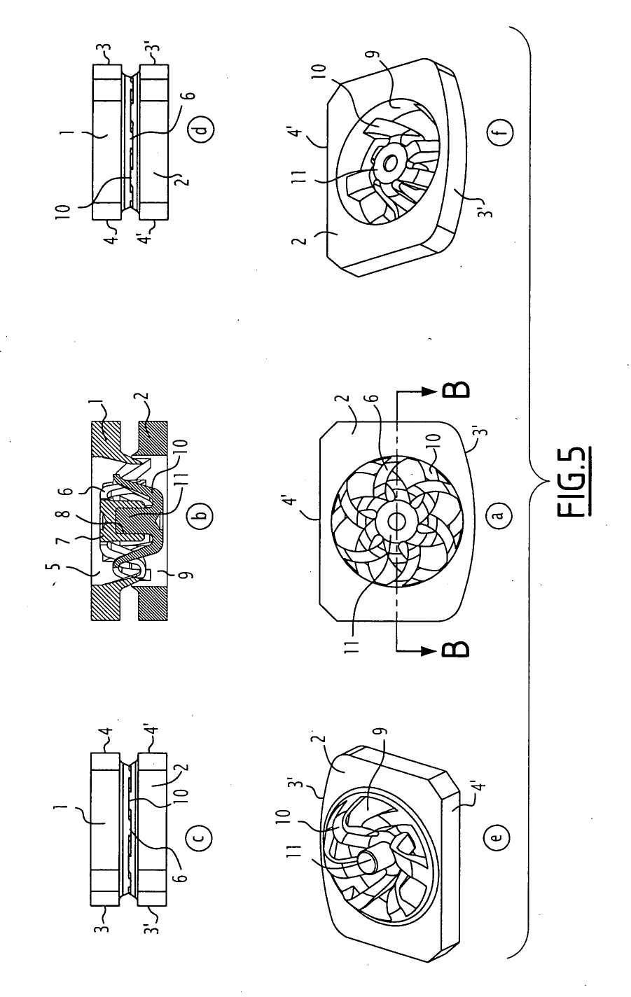

The implant shown in FIG. 5 is similar to that shown in FIG. 4, except that the webs 6, 10 are thinner and have a more accentuated curvature, to the extent that when the implant is assembled, the webs 6 of the upper plate 1 and the webs 10 of the lower plate 2 are interlaced. So, the useful fiber length for flexion can be increased, and the displacement of the webs 6, 10 in compression can be optimized.

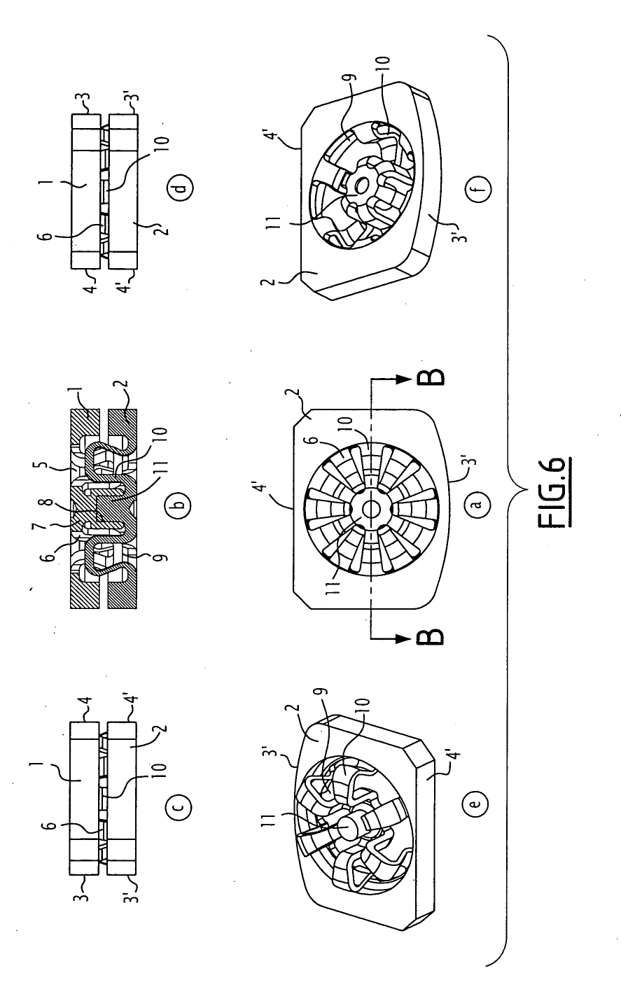

The implant shown in FIG. 6 is similar to that of FIG. 1 except that the webs 6, 10 have a cross section approximately Ω shape. So, the useful fiber length for flexion can be increased, and the displacement of the rotation center can be optimized.

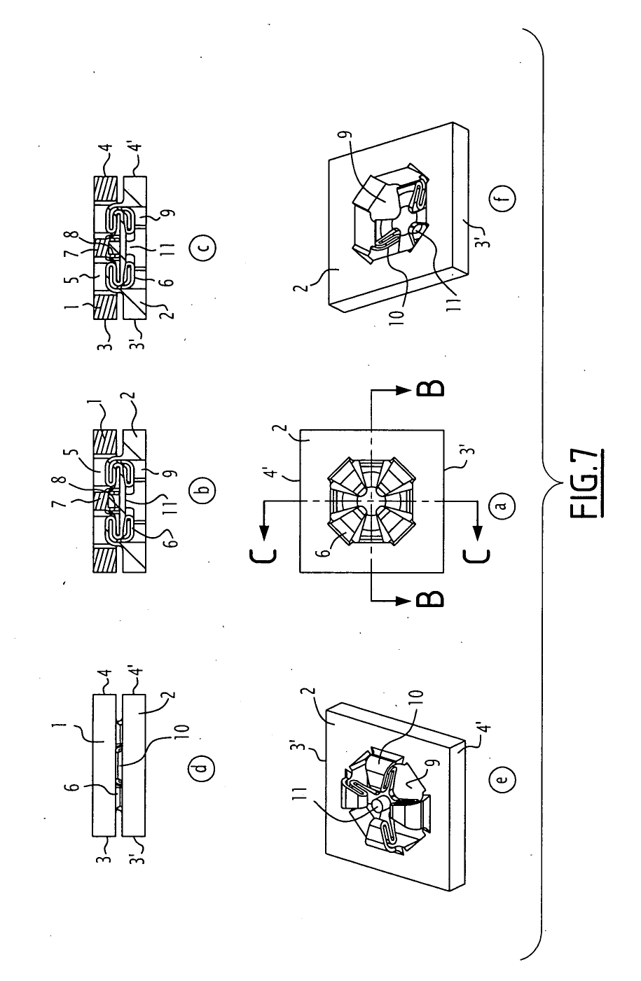

The implant shown in FIG. 7 is different from the preceding ones in that the outer shape of the plates 1, 2 is a simple square. In this case, there is no particular attempt to follow the contours of the vertebral plates between which the implant is to be installed. This configuration could be adopted for the other implants which have been or will be described. Moreover, the implant of FIG. 7 is also distinguished from that of FIG. 1 in that the webs 6, 10 have an S-shaped cross section. So, the useful fiber length for flexion can be very substantially increased.

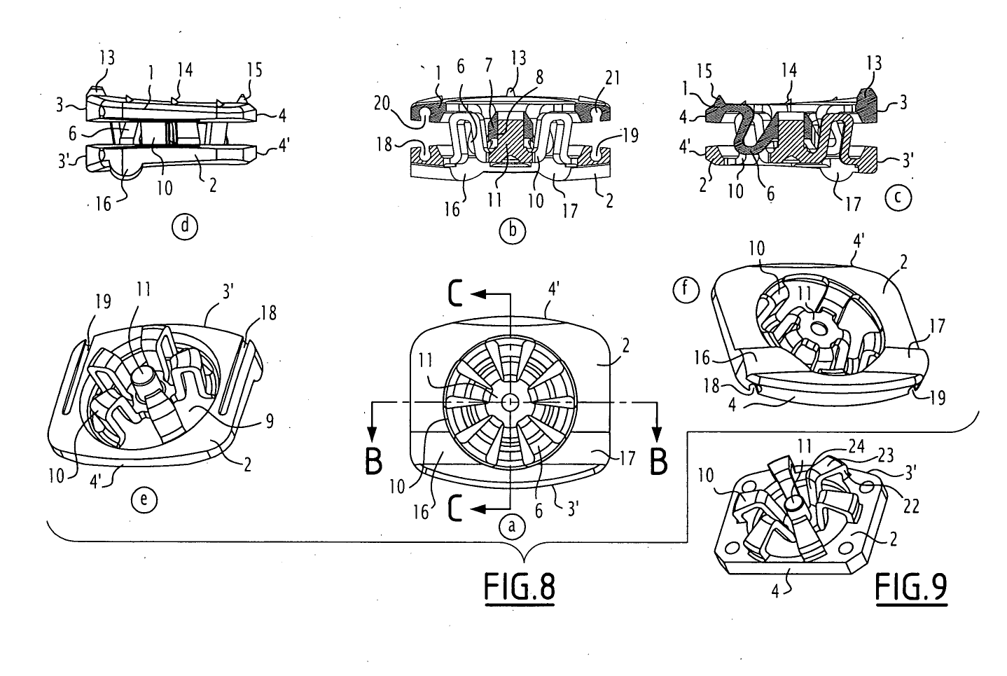

The implant shown in FIG. 8 resembles that of FIG. 2 in that the webs 6, 10 are U-shaped. However, it will be noted that the opening of the U is slightly inclined toward the interior of the plates 1, 2, and more strongly so for the upper plate 1 than for the lower plate 2. Another difference from the preceding implants is the configuration of the plates 1, 2 themselves. The upper plate 1 has on its face intended to come into contact with the vertebral plate a slightly concave shape longitudinally and a slightly convex shape transversely. It also has pointed excrescences on its perimeter, intended to facilitate anchoring of the implant to the vertebral plate of the upper vertebra. The lower plate 2 has, on its face intended to come into contact with the vertebral plate, two excrescences in the form of transverse bosses. These bosses 16, 17 get inserted into the patient's vertebral plates. To this end, when the vertebral plates are prepared before the insertion of the implant, female recesses are bored in the vertebral plates, the shapes of which correspond to the shapes of the bosses 16, 17. The two plates 1, 2 also have two longitudinal grooves (18, 19; 20, 21) in their internal faces. These grooves (18, 19; 20, 21) are used for connecting the implant-carrier when the implant is inserted by impaction. It is, so, possible to make the implant/implant carrier assembly be a rigid block, which is likely not to undergo mechanical stresses during the insertion of the implant.

FIG. 9 shows an alternative of the lower plate 2. Differing from other configurations of the webs 10 described up to now, its webs 10 are constituted by successive rectilinear portions 22, 23, 24 and not by curved portions. They are connected to one another practically at a right angle (in the shown example; this is not compulsory) and form a U open toward the center of the plate 2. This type of lower plate 2 may be associated with an upper plate 1 (not shown) whose webs 6 are configured in the same way or differently.

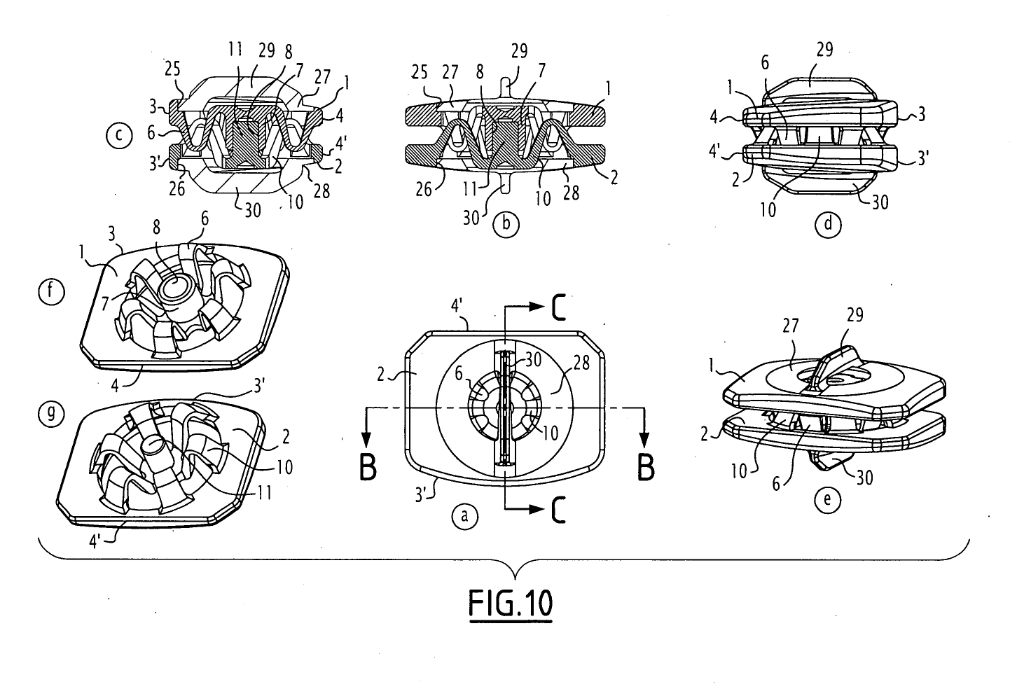

FIG. 10 shows an implant which differs from the alternative of FIG. 1 essentially in the external configuration of the external faces of the upper 1 and lower 2 plates. These plates 1, 2 have biconvex external surfaces, i.e. are convex in both the longitudinal and the transverse direction. On the other hand, each of the plates 1, 2 comprises on its external surface a circular cavity (in the example shown) 25, 26 within which a cover 27, 28 of corresponding shape is lodged and is fixed by means not shown (screws, laser weld, cement, for example). These covers 27, 28 each bear an excrescence 29, 30 extending along the longitudinal axis of the plate 1, 2 and intended to form an anchoring element for the implant in the corresponding vertebral plate.

Generally, it should be understood that, in this class of alternatives, there is no obligation for the plates 1, 2 to have webs of similar configurations. If it is desired to confer different deformation characteristics on the plates 1, 2, webs of different conformations may be used, on condition, of course, that their geometries are compatible: the webs of a given plate should be able to be intercalated between the webs of the other plate without mutual contact. Similarly, the external faces of the plates 1, 2 may have different configurations.

The described and shown examples are not limitative, either, as for the number of webs 6, 10 of each plate. It can be higher or lower than what is shown on the drawings.

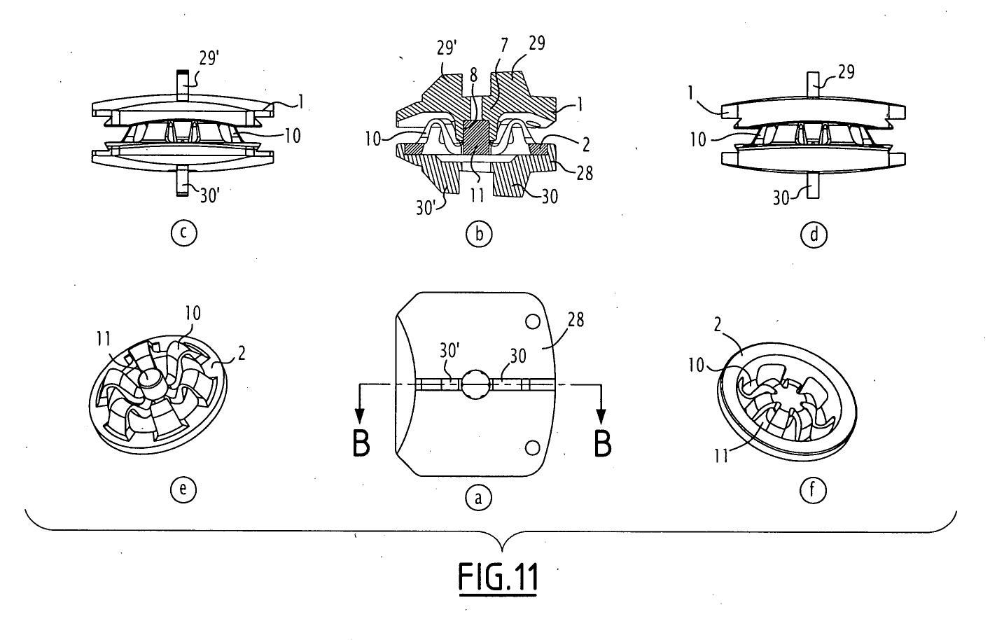

In the embodiment shown on FIG. 11, a lower plate 2 provided with webs 10 similar (in the shown example) to the webs of the embodiment of FIG. 1, is associated to an upper plate 1 which is totally deprived of webs, and so entirely rigid. In this example, it can also be noted that the lower plate 2 has a circular periphery, and that it is inserted in its cover 28, which, accordingly, confers to the implant its external shape. In the example of FIG. 10, on the contrary, the covers 27, 28 are inserted in the plates 1, 2. Moreover, the single longitudinal excrescences 29, 30 are each replaced by two longitudinal excrescences (29, 29′; 30, 30′), which are interrupted in the central zone of the cover 27, 28 which bears them. These features can be adapted to other types of implants.

The preceding examples of implants are characterized by the fact that the two plates, upper 1 and lower 2, are originally separate parts, which are assembled at the level of their hubs 7, 11. In the following examples, the two plates become integral and are joined at the level of a socket. But all the functionalities described for the preceding examples are found, or may be found, in the examples which follow.

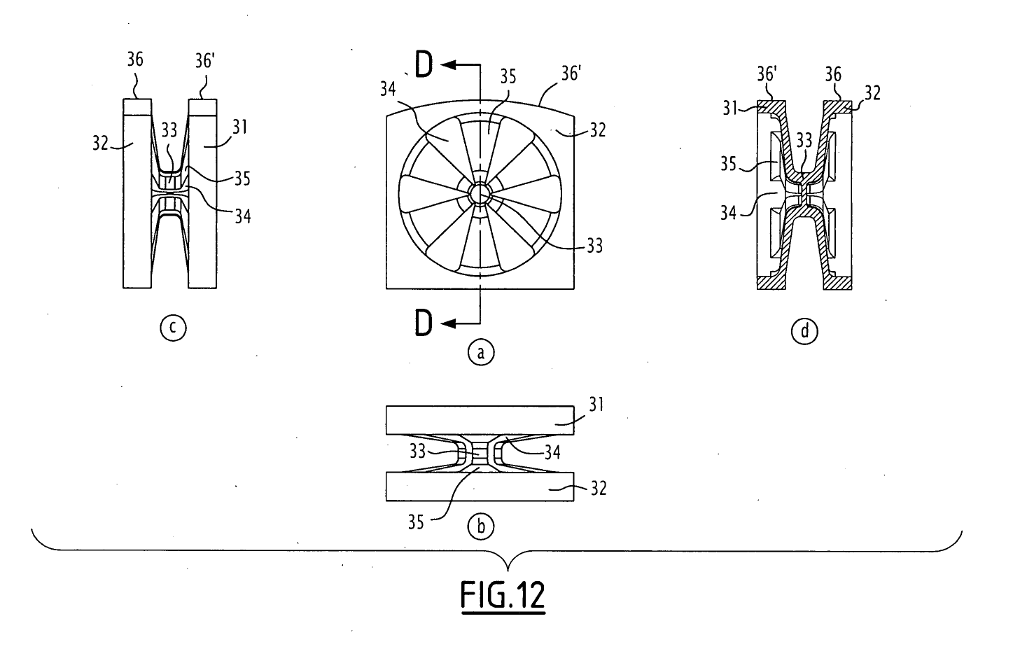

The example of an implant shown in FIG. 12 is composed, upon manufacture, of a single part having an upper pate 31 and a lower plate 32, connected at the level of a socket 33 which, in the example shown, is located substantially at the center of the implant. Orifices 34 are cut out in the plates 31, 32 so that the remaining material forms webs 35 connecting the periphery of the plates 31, 32 to the socket 33. In the example of FIG. 12, these webs 35 are six in number, identical and regularly distributed around the socket 33, which is here of approximately cylindrical shape. In the example shown, the front edges 36, 36′ of the plates 31, 32 are rounded so as to closely follow the shape of the vertebral plates in the zone where the implant is to be inserted.

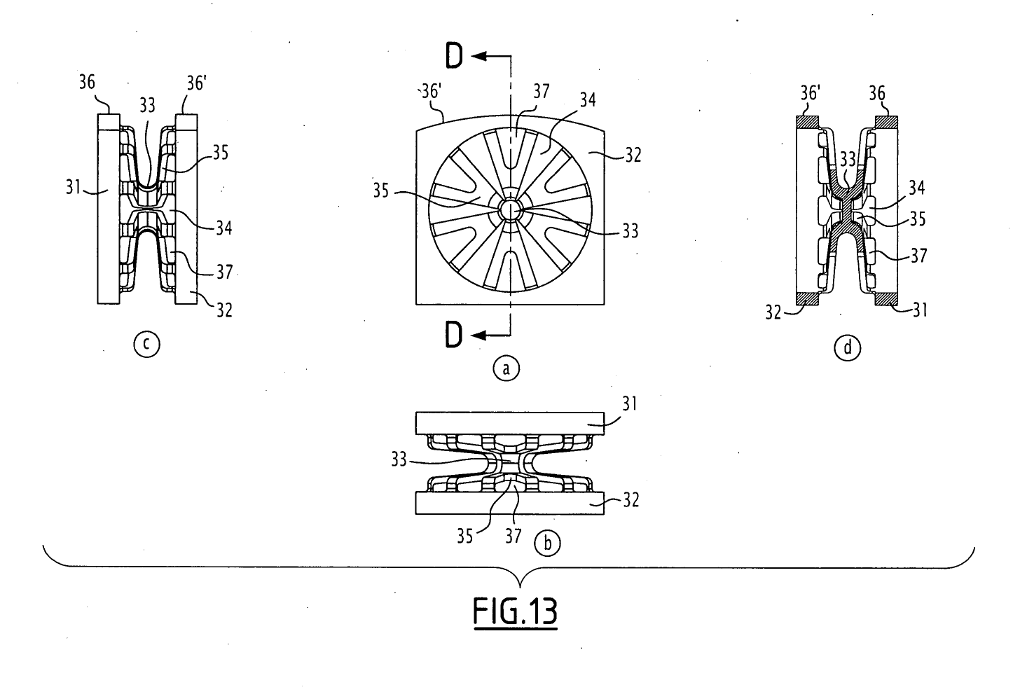

The example of an implant shown in FIG. 13 is analogous to that of FIG. 12, except that in the example of FIG. 3, cavities 37 have been created in the webs 35 so that each web is attached to the socket 33 at a single point, and to the periphery of the plate 31 or 32 at two points. The flexibility of the webs 35 is thus increased.

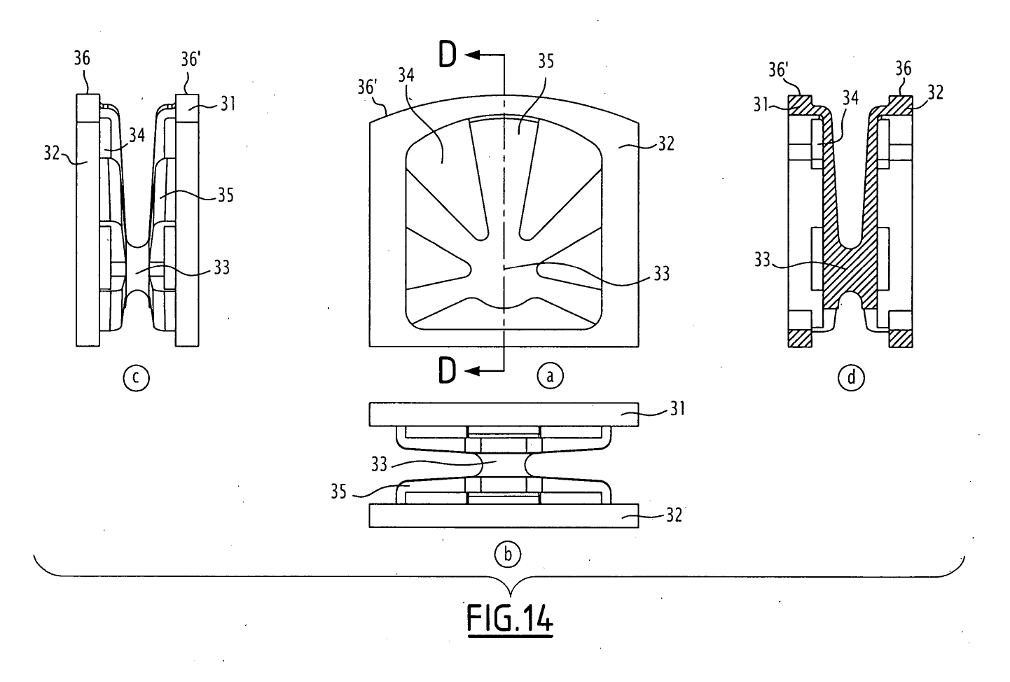

The implant shown in FIG. 14 is similar to that of FIG. 12 in principle, except that the socket 33 is no longer at the center of the implant, but is offset on the longitudinal axis of the implant toward the rear zone. The webs 35 are then found with different lengths, widths and thicknesses. Furthermore, in the example shown, there are only five webs 35 because none was provided for connecting the socket 33 and the rear edges of the plates 31, 32 along the longitudinal axis of the implant. Such a web would inevitably be very short, and thus of little flexibility, and would considerably limit the deformation capacity of the implant.

Because of the rearward offset of the socket 33 with respect to the center of the implant, a better reproduction is attained of the natural division of forces undergone and exerted by the disk during the patient's movements. In particular, the implant is endowed with the possibility of a deformation of greater amplitude when the rachis works in flexion (when the patient leans forward) than when the rachis works in extension (when the patient leans backward).

A forward offset of the socket 33 can also be considered, for cases where one would wish, for example, to favor a possibility of extension movements at the concerned level of the rachis. Generally, the location of the socket 33 can be optimally chosen according to the patient's morphology and weight, and also according to the level of the rachis where the implant is to be inserted.

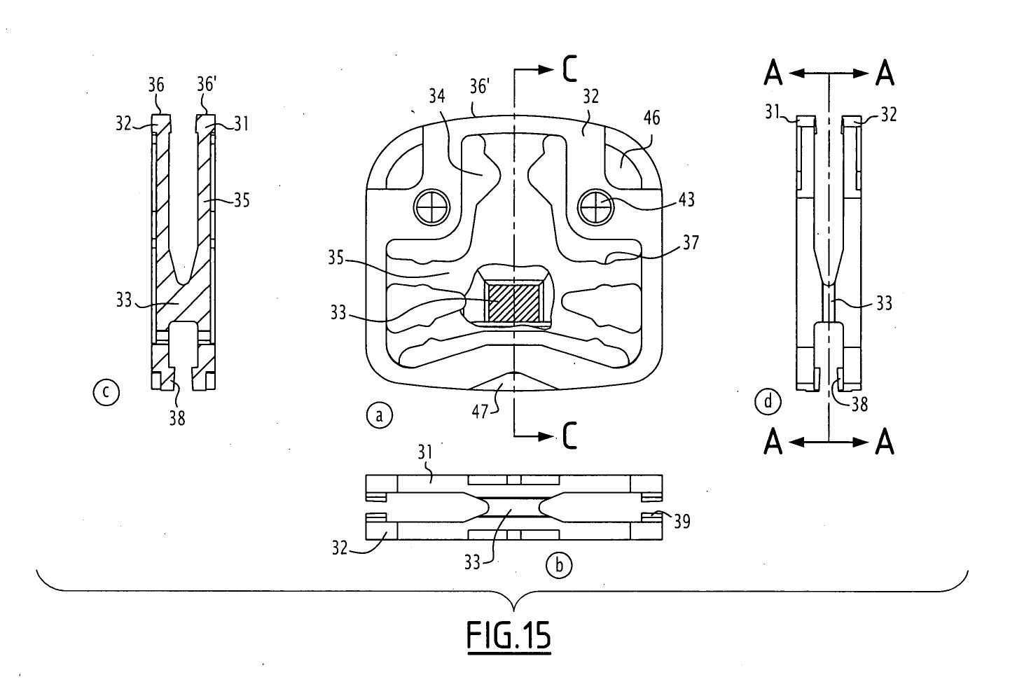

The implant shown in FIG. 15 is comparable to that of FIG. 14. It differs from it essentially in the following points. Firstly, the socket 33 has a rectangular transverse section, not circular. In this way, deformations of the implant in lateral directions may be privileged, if as shown the large dimensions of the rectangle are perpendicular to the longitudinal axis of the implant. On the other hand, the edges of the webs 35 are no longer rectilinear, but have indentations 37 which thin them locally and increase their flexibility.

It is also seen that, differing from the configuration of FIG. 14, the webs 36 are located in the same plane as the plates 31, 32, simplifying the construction of the whole. Stops 38, 39 have also been provided on the mutually facing edges of the plates 31, 32 , and limit the amplitude of relative movements of the plates 31, 32 in the case of extension of the rachis (stops 36) and when the patient leans to the side (stops 38).

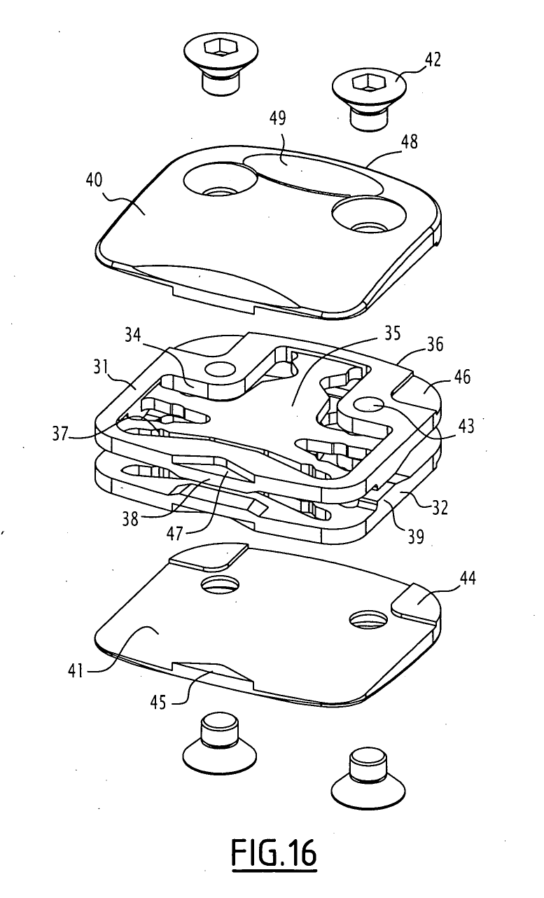

Preferably, as shown in FIG. 16, this implant has each of its plates 31, 32 equipped with a cover 40, 41. These covers 40, 41 ensure the contact between the implant and the vertebral plates. They preferably have a biconvex outer surface for a better contact. They are fixed to the plates 31, 32 by screws 42 penetrating into threaded holes formed in the plates 31, 32. A connection by peripheral cementing and/or welding (laser or TIG) at the level of the contacting zones between the plates 31, 32 and the covers 40, 41 can also be added or substituted to the connection by screws 42. Protrusions 44, 45 formed in the surfaces of the covers 40, 41 which contact the implant are inserted into hollows 46, 47 corresponding to the plates 31, 32 to ensure good positioning of the covers 40, 41. The covers 40, 41 also have a boss 49, the function of which is similar to the function of the aforesaid bosses 16, 17, in the neighborhood of their front edge 48.

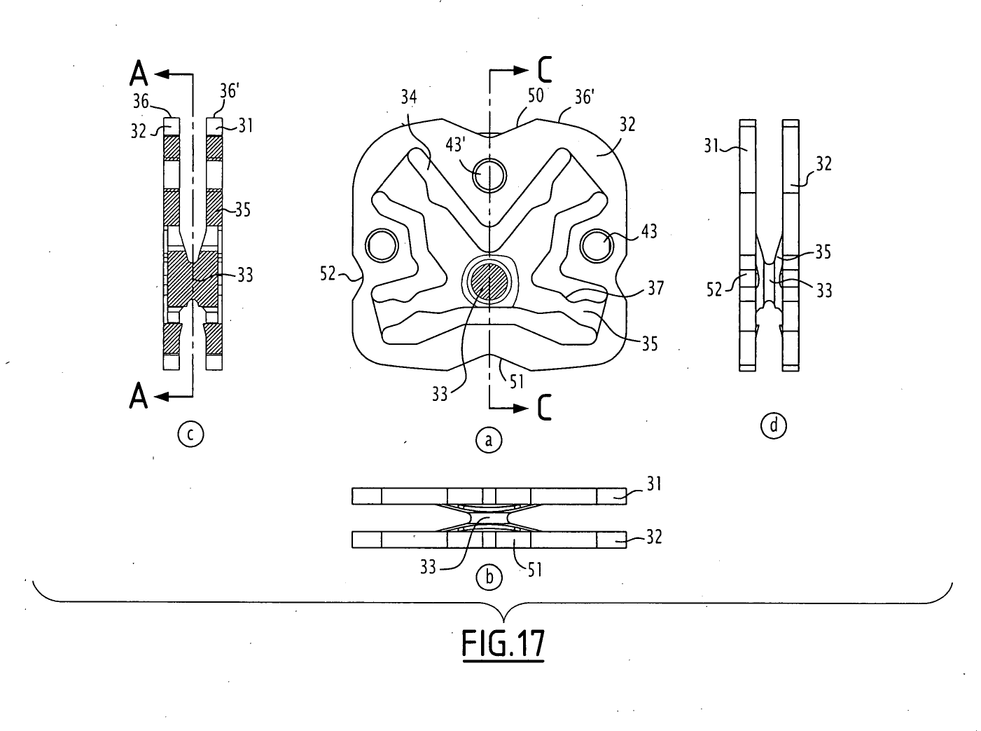

The implant shown in FIG. 17 is comparable to that of FIGS. 15 and 16. It particularly differs from it in that the socket 33 has a circular cross section, and in that there are no more than four webs instead of five. There is thus room to form in each plate 31, 32 a third threaded hole 43′ located on the longitudinal axis of the implant for a fixing screw 42 of a cover 40, 41. It is, so, possible to obtain a better repartition of the anchoring points which, preferably in combination with a welding and/or a cementing, gives a better strength to the assembly. Indentations 50, 51, 52 are likewise formed in the edges of the plates 31, 32 for the passage of stops provided on the covers 40, 41.

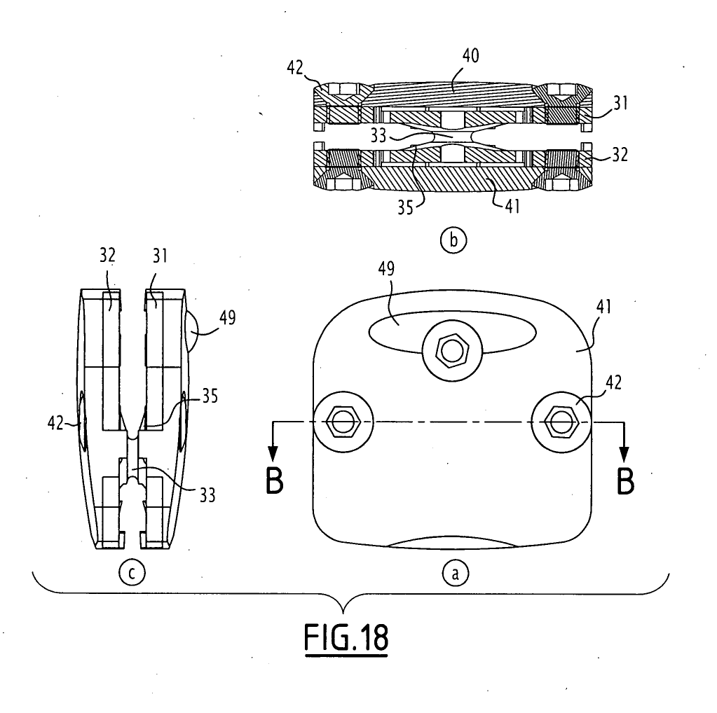

FIG. 18 shows this same implant equipped with its covers 40, 41 comparable to those of the implant of FIGS. 15 and 16.

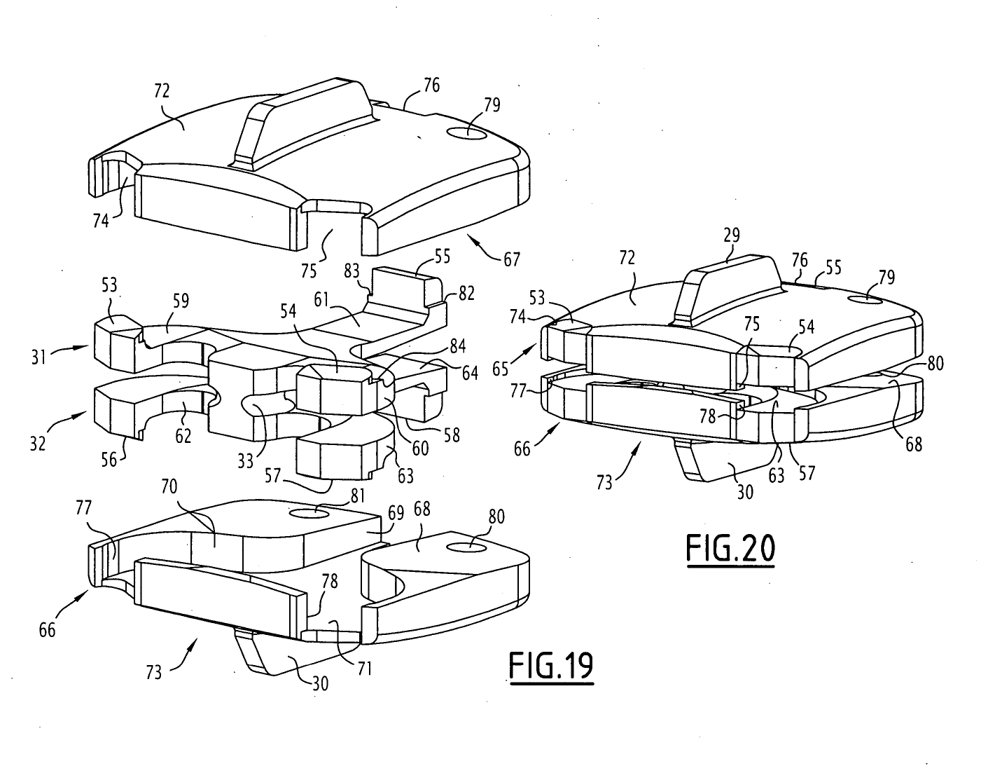

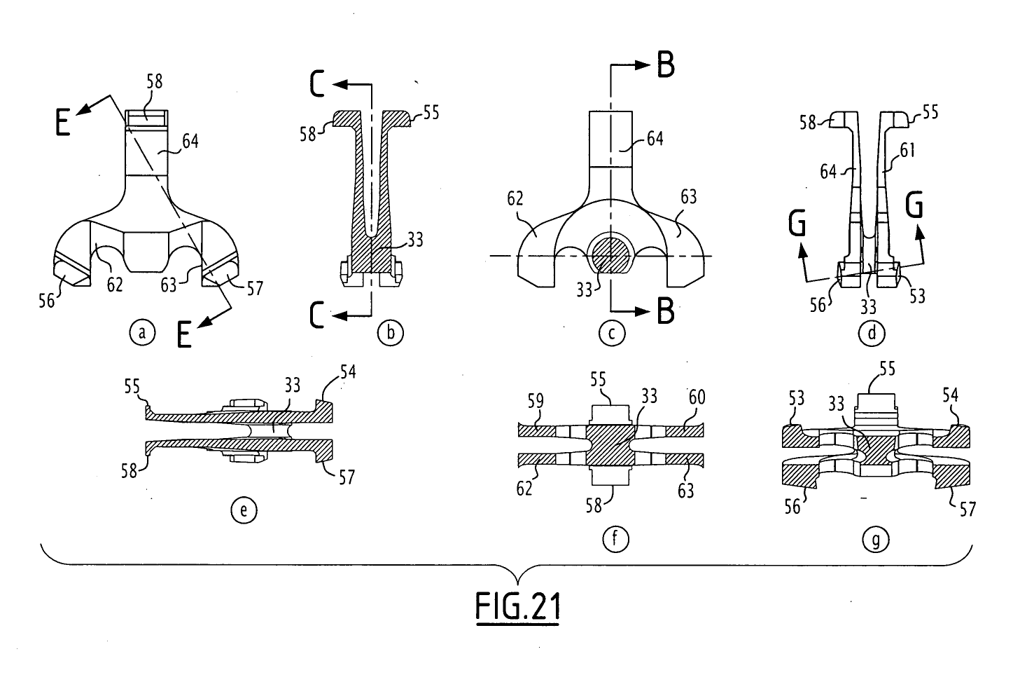

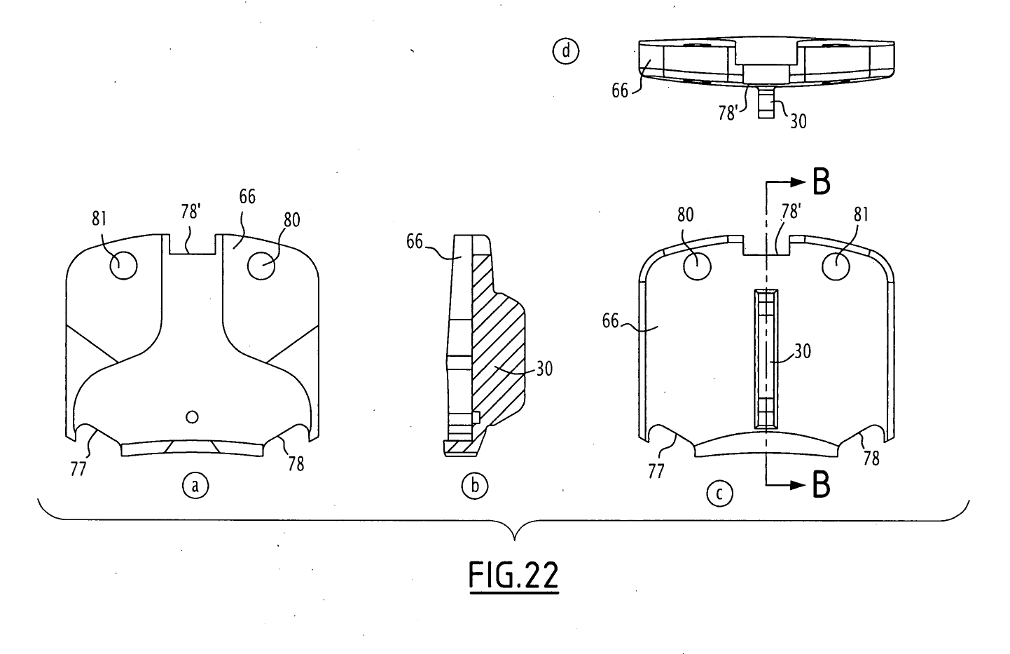

FIGS. 19 and 20 show the disassembled state (FIG. 19) and the assembled state (FIG. 20) of another example of an implant. FIGS. 21 and 22 show more in detail the components of the implant.

In this example, the portions of the plates 31, 32 of the preceding examples which come into contact with the vertebral plates are each reduced to three bearings (53, 54, 55; 56, 57, 58) connected to the socket 33 (which has a truncated circular cross section here; it could have a rectangular cross section) by webs (59, 60, 61; 62, 63, 64). In fact, this comes to having extended the cutouts 34 of the preceding examples so as to have them include the edges of the plates 31, 32.

Two bearings (53, 54; 58, 57) are placed at the two extremities of the rear edge of each plate 31, 32, and the portions of the webs (59, 60; 62, 63) which connect them to the socket 33 have in the shown example a shape approximately of a quarter circle or of a quarter ellipse. The other bearing 55, 58 of each plate 31, 32 is placed at the middle of the front edge of the corresponding plate 31, 32 and is connected to the socket 33 by a web 61, 64 which substantially follows the longitudinal axis of the plate 31, 32.

The implant is completed by two covers 65, 66 which are each placed on an upper or lower face of one of the plates 31, 32. The internal faces 67, 68 of the covers 65, 66 bear grooves 69, 70, 71 into which the webs (59, 60, 61; 62, 63, 64) are inserted without any contact. The covers 65, 66 also have indentations (74, 75, 76; 77, 78, 78′) on the periphery of their external face 72, 73, enabling the bearings (53, 54, 55; 56, 57, 58) to be brought level, that is to themselves form portions of the external surfaces of the implant once assembled, being located in the exact prolongation of the external faces 72, 73 of the covers 65, 66.

Preferably, the external faces 72, 73 of the covers 65, 66 bear excrescences 29, 30 ensuring the anchoring of the implant to the vertebral plates. These excrescences 29, 30 may take various forms, particularly those described and shown for other examples of implants.

The covers 65, 66 also preferably have orifices 79, 80, 81 for the insertion of gripping instruments facilitating the insertion of the implant. Such orifices 79, 80, 81 may be found in the other examples of implants which have been described.

Preferably, shoulders 82, 83, 84, 85 are provided on the webs (59, 60, 61; 62, 63, 64) near the bearings (53, 54, 55; 56, 57, 58), so as to ensure a limitation of the penetration of the webs (59, 60, 61; 62, 63, 64) in the grooves 69, 70, 71 of the covers 65, 66. One is, so, more secure that contacts between the webs (59, 60, 61; 62, 63, 64) and the walls of the grooves 69, 70, 71 will be avoided.

The covers 65, 66 preferably have, as shown, a biconvex external surface 72, 73, and the bearings (53, 54, 55; 56, 57, 58) consequently have shapes which prolong those of the covers 65, 66.

The implant which has been described and shown in FIGS. 19, 20, 21, 22 could of course be modified, for example by providing more than three bearings (53, 54, 55; 56, 57, 58) per plate. The implant may particularly be assembled by laser welding.

This configuration shown in FIGS. 19 to 22 gives an excellent compromise between the deformation and strength properties of the implant and its manufacturing cost.

The different elements of the implants according to different embodiments of the invention may particularly be manufactured by electro-erosion, laser cutting or molding. They may particularly be made of titanium or carbon fibers.

The different implants which have been described and shown are non-limiting examples. A characteristic may be taken from one of the examples and transposed into another of the examples, in place of another functionally equivalent characteristic, or in addition to other described characteristics, if this appears possible to those skilled in the art, without departing from the scope of the invention.

Because of the absence of mechanical connecting parts with displacement, the implants according to some of the present embodiments do not generate debris which may become spread in the organism of the patient.

These implants can be used, for example, in two different ways. In a first way, they can be a total intervertebral disk prosthesis. The surgeon performs a total discectomy of the damaged disk, then an adequate preparation of the vertebral plates by conventional instruments and techniques. Then, the implant is placed in position by the anterior approach, again by conventional methods.

In a second way, they can be used as elements of a rachis stabilization device. To this end, they may replace only a part of the disk. They are, in a manner which is known as for intervertebral implants, associated with other rachis stabilizing elements attached to the vertebrae of the region to be stabilized. The main elements known to this end include one or several rods which longitudinally stretch along the rachis, and are attached to the vertebrae, for example by means of pedicle screws. In such an embodiment, one ore several implants of the type which has been described can be used, according to the number of intervertebral disks included in the region of the rachis to be stabilized.

In some embodiments of the stabilization device, it is possible to use intervertebral implants which may not be all identical, the main point being that one of them, at least, is of the type which has been described.

As will have been seen, in the implant of one or more embodiments of the invention, the two plates intended to be placed in contact with the vertebral plates are shaped so as to provide them with a significant capacity for elastic deformation and flexibility, as well as a mobile center of rotation. For this purpose, the plates have a cut-out which delineates elastic webs converging toward a solid zone where the plates are connected to one another. The deformations of the elastic webs make possible a great variety of relative movements between the vertebrae, namely:
an axial compression,
an anterior/posterior flexure,
a lateral flexure,
an axial rotation,
combinations of all these movements;
a mobility of the instantaneous center of rotation.
   The behavior of the natural disks is reproduced to the best extent.

In a first series of alternatives of the implant, the two plates are distinct in construction and nest one into the other at the level of their solid zones, for example by means of a connection by a tight fit and/or by means of cement and/or by welding (TIG or laser).

In a second series of alternatives of the implant, the two plates form a unit and are united at the level of a socket which may be located substantially in a central position or in an offset position toward the rear or front region on the longitudinal axis of the implant.

The webs may not be in mutual contact when the implant is ready for use. In particular, if the webs are not planar, the webs of the upper and lower plates have to be intercalated one between another to avoid such a contact which would prevent satisfactory functioning of the implant.

This implant can be used, for example, in two different ways. It can be used as a total intervertebral disk prosthesis. To this end, the surgeon performs a total discectomy, then inserts the implant of one or more embodiments of the invention between the involved vertebrae. It can also be used for replacing, possibly only partially, an intervertebral disk, in combination with a classical rachis stabilization device, in particular a device including longitudinal rods, attached to the vertebrae for example by means of pedicle screws. Other ways of use may also exist.

Although only a few exemplary embodiments of this invention have been described in detail above, those skilled in the art will readily appreciate that many modifications are possible in the exemplary embodiments without materially departing from the novel teachings and advantages of this invention. For example, although the teachings have been directed to the intervertebral space, other embodiments of the present invention can be directed to other areas, including the knee, the femur, and so forth. Accordingly, all such modifications are intended to be included within the scope of this invention as defined in the following claims.

We claim:

1. An intervertebral implant, comprising two connected plates configured to contact with opposing vertebral plates, wherein at least one of the plates has a cut-out delineating webs converging toward a zone where the plates are connected to one another, the webs not being in mutual contact when the implant is ready for use.

2. The implant of claim 1, wherein the plates are initially separate pieces, one of the plates having, in its convergence zone of the webs, a hub comprising a seat, and the other of the plates having, in its convergence zone of the webs, a hub comprising an excrescence able to penetrate and be kept in said seat for fixing said plates to one another.

3. The implant of claim 1, wherein the plates are two portions of the same part and are connected to one another by a socket.

4. The implant of claim 3, wherein the socket has a circular transverse cross section.

5. The implant of claim 3, wherein the socket has a rectangular transverse cross section.

6. The implant of claim 1, wherein the webs of at least one of the plates have a substantially V-shaped longitudinal cross section.

7. The implant of claim 1, wherein the webs of at least one of the plates have a substantially U-shaped longitudinal cross section.

8. The implant of claim 7, wherein the opening of the U-shape is inclined toward an interior of the plates.

9. The implant of claim 1, wherein the webs of at least one of the plates have a cross section substantially in the shape of an Ω.

10. The implant of claim 1, wherein the webs of at least one of the plates has a longitudinal cross section of substantially S shape.

11. The implant of claim 1, wherein the webs of at least one of the plates are formed by a succession of rectilinear portions.

12. The implant of claim 1, wherein the webs of at least one of the plates are located in a same plane as the plate.

13. The implant of claim 1, wherein the webs of at least one of the plates have a cavity.

14. The implant of claim 1, wherein the webs of at least one of the plates have an orientation having a helicoidal curvature.

15. The implant of claim 14, wherein the webs of one plate are interlaced with the webs of the other plate.

16. The implant of claim 1, wherein the zone where the plates are connected to one another is located substantially at a center of the implant.

17. The implant of claim 1, wherein the zone where the plates are connected to one another is located on a longitudinal axis of the implant and is offset into a posterior zone of the implant.

18. The implant of claim 1, wherein the zone where the plates are connected to one another is located on a longitudinal axis of the implant and is offset into an anterior zone of the implant.

19. The implant of claim 1, wherein at least one external face of the implant has a longitudinally concave shape and a transversely convex shape.

20. The implant of claim 1, wherein at least one external face of the implant has a biconvex form.

21. The implant of claim 1, wherein at least one external face of the implant has at least one excrescence for anchoring the implant to a corresponding vertebral plate.

22. The implant of claim 1, wherein an external face of at least one of said plates has a cover.

23. The implant of claim 1, wherein the webs have indentations.

24. The implant of claim 1, wherein the plates have stops on their facing edges.

25. The implant of claim 3, wherein each plate comprises a cover and at least three bearings connected to the socket by the webs, said bearings being located in a prolongation of an external surface of the respective cover having indentations enabling said bearings to be brought level to the external surface, the covers having grooves on an internal surface for lodging the webs.

26. The implant of claim 25, wherein the bearings are three in number for each plate; in that one of the bearings is located in a middle of an anterior edge of the corresponding plate, the web which bears it substantially following a longitudinal axis of the implant.

27. The implant of claim 26, wherein the other two bearings are connected to the socket by webs having a shape approximately of a quarter circle or of a quarter ellipse.

28. An intervertebral implant, comprising two connected plates configured to contact with opposing vertebral plates, wherein each plate has a cut-out delineating webs converging toward a zone where the plates are connected to one another, and a hub comprising a seat and an excrescence able to penetrate and be kept in the seat for fixing said plates to one another.

29. The implant of claim 28, wherein the webs of one plate are interlaced with the webs of the other plate.

30. The implant of claim 29, wherein each plate includes an external, concaval surface and a cover.

25. An intervertebral implant, comprising:
two connected plates configured to contact with opposing vertebral plates;
a plurality of webs converging toward a zone where the plates are connected to one another; and
a cover for each plate.

26. The intervertebral implant of claim 25 wherein each cover is configured to communicate with one or more bearings connected to the socket by the webs, said bearings being located in an external surface of the respective cover.

27. The intervertebral implant of claim 26, wherein the bearings are at least three in number for each plate; in that one of the bearings is located in a middle of an anterior edge of the corresponding plate, the web which bears it substantially following a longitudinal axis of the implant.
